(19) United States
(12) Patent Application Publication
(10) Pub. No.: US 2022/0108787 A1
(43) Pub. Date: Apr. 7, 2022

(54) DEVICE AND METHOD FOR MANAGING MEDICAL IMAGE DATA AND TREATMENT DATA

(71) Applicant: GENORAY CO., LTD., Seongnam-si (KR)

(72) Inventors: Young Hyun Lim, Seongnam-si (KR); Young Hwan CHOI, Seongnam-si (KR)

(21) Appl. No.: 17/416,272

(22) PCT Filed: Nov. 14, 2019
(86) PCT No.: PCT/KR2019/015540

§ 371 Date: Dec. 23, 2021

(30) Foreign Application Priority Data
Dec. 18, 2018 (KR) 10-2018-0164496

(51) Int. Cl. G16H 30/40; G06K 9/62; G06F 16/538

(52) U.S. Cl. CPC G16H 30/40; G06F 16/538; G06K 9/628

(57) ABSTRACT

The present invention provides an apparatus for managing medical image data and treatment data, including a database for storing medical image data of a patient imaged by a plurality of imaging devices and treatment data generated based on the medical image data, a data processing unit for generating thumbnails of the medical image data and the treatment data, and classifying and generating a plurality of mounts for storing the thumbnails for each imaging device, and a display unit for displaying the plurality of mounts and the thumbnails stored in the plurality of mounts.

### CROSS-REFERENCE TO RELATED APPLICATION

This application claims priority to and the benefit of Korean Patent Application No. 10-2018-0164496, filed on Dec. 18, 2018, the disclosure of which is incorporated herein by reference in its entirety.

### BACKGROUND

### 1. Field of the Invention

The present invention relates to an apparatus and method for managing medical image data and treatment data, and more particularly, the present invention relates to an apparatus and method for managing medical image data and treatment data that can comprehensively compare image data and treatment data for each imaging device and patient on one screen.

### 2. Discussion of Related Art

The operator establishes a treatment plan and designs a treatment device based on the patient's 2D medical image data and 3D medical image data, which are captured using a plurality of imaging devices.

Currently, since commercially available medical devices can analyze only one medical image data for each imaging device, it is impossible to comprehensively compare and analyze medical image data for a plurality of imaging devices on one screen.

In addition, for currently available medical devices in the market, the process of generating medical image data by imaging a patient by an imaging device includes selecting a patient to be imaged, selecting an imaging device, and generating medical image data by imaging the selected patient with the selected imaging device. However, since such a process of generating medical image data undergoes a number of complicated processes, operator errors frequently occur, and there is a problem in that the convenience of use is lowered.

In addition, when the image data is not located in the desired location of the operator for various reasons such as setting of imaging devices, file loading policy, the operator's mistakes and the like, it is not possible to facilitate moving and copying of the image data between the imaging devices and the patients.

As such, there is a problem in that currently available medical devices in the market cannot provide the operator with intuitiveness and convenience of comparing medical images.

### SUMMARY OF THE INVENTION

An object of the present invention is to provide an apparatus and method for managing medical image data and treatment data that can comprehensively compare and analyze medical image data and treatment data for each imaging device and patient.

In addition, another object of the present invention is to provide an apparatus and method for managing medical image data and treatment data, which can minimize an image capturing step to prevent an operator's error and provide the convenience of use.

In addition, another object of the present invention is to provide an apparatus and method for managing medical image data and treatment data that can facilitate moving and copying of data between patients on one screen.

In addition, another object of the present invention is to provide an apparatus and method for managing medical image data and treatment data that can set a customized layout according to the treatment site and treatment plan that vary for each patient.

[Korea National R&D Project that supported this invention]
Project Assignment Number: 10077361
Name of Ministry: Ministry of Trade, Industry and Energy (MOTIE, Korea)
Research Management Professional Institution: Korea Evaluation Institute of Industrial Technology (KEIT, Korea)
Research Program Name: Advanced Technology Center (ATC) Program
Research Project Name: Integrated System for Dental Diagnosis, Treatment Simulation & PSI (Patient Specific Instrument) Design
Host Institution: Genoray Co., Ltd.
Research Period: Apr. 1, 2017 to Dec. 31, 2021

In order to achieve the aforementioned objects, the present invention provides an apparatus for managing medical image data and treatment data, including a database for storing medical image data of a patient imaged by a plurality of imaging devices and treatment data generated based on the medical image data, a data processing unit for generating thumbnails of the medical image data and the treatment data, and classifying and generating a plurality of mounts for storing the thumbnails for each imaging device, and a display unit for displaying the plurality of mounts and the thumbnails stored in the plurality of mounts.

Herein, when a first mount selection signal for a first selection mount selected from the plurality of mounts and an imaging signal for imaging the patient are received, the data processing unit transmits imaging device information for the imaging device including the first selection mount.

In addition, it further includes an imaging control unit for receiving the imaging device information and controlling the imaging device selected based on the imaging device information to image the patient.

In addition, the imaging control unit receives the medical image data generated by the selected imaging device imaging the patient and transmits the same to the data processing unit and the database.

In addition, the data processing unit generates a first thumbnail of the medical image data received from the imaging control unit, and stores the first thumbnail in the first selection mount.

In addition, when a second mount selection signal for a second selection mount selected from the plurality of mounts and a loading signal for loading the image data or treatment data are received, the data processing unit loads the medical image data or the treatment data from the database.

In addition, the data processing unit generates a second thumbnail of the medical image data or the treatment data loaded from the database, and stores the second thumbnail in the second selection mount.

In addition, when a third mount selection signal for a third selection mount selected from the plurality of mounts and a copy signal for copying a third thumbnail stored in the third selection mount or a move signal for moving the third thumbnail are received, the data processing unit stores the third thumbnail. Also, when a fourth mount selection signal for a fourth selection mount selected from the plurality of mounts and a paste signal for pasting the third thumbnail are received, the data processing unit stores the third thumbnail in the fourth mount.

In addition, it further includes a screen configuration unit for setting the position, size and number of the mounts for each patient and imaging device.

In addition, the present invention provides a method for managing medical image data and treatment data, including storing medical image data of a patient imaged by a plurality of imaging devices and treatment data generated based on the medical image data in a database, generating thumbnails of the medical image data and the treatment data, and classifying and generating a plurality of mounts for storing the thumbnails for each imaging device, storing the thumbnails in the plurality of mounts, and displaying the plurality of mounts and the thumbnails stored in the plurality of mounts.

In addition, it further includes receiving a first mount selection signal for a first selection mount selected from the plurality of mounts and an imaging signal for imaging the patient, transmitting imaging device information for the imaging device comprising the first selection mount, receiving the imaging device information and controlling the imaging device selected based on the imaging device information to image the patient, transmitting the medical image data generated by imaging the patient by the selected imaging device, and generating a first thumbnail of the received medical image data and storing the first thumbnail in the first selection mount.

In addition, it further includes receiving a second mount selection signal for a second selection mount selected from the plurality of mounts and a loading signal for loading the image data or treatment data, loading the medical image data or the treatment data from the database according to the loading signal, and generating a second thumbnail of the medical image data or the treatment data loaded from the database, and storing the second thumbnail in the second selection mount.

In addition, it further includes storing a third thumbnail when a third mount selection signal for a third selection mount selected from the plurality of mounts and a copy signal for copying the third thumbnail stored in the third selection mount or a move signal for moving the third thumbnail are received, and storing the third thumbnail in a fourth mount when a fourth mount selection signal for a fourth selection mount selected from the plurality of mounts and a paste signal for pasting the third thumbnail are received.

In addition, it further includes setting the position, size and number of the mounts for each patient and imaging device.

According to the present invention, by classifying medical image data and treatment data for each imaging device and displaying the same as thumbnails on a display unit, it is possible to comprehensively compare and analyze medical image data and treatment data for each imaging device on one screen, and thus, it has an effect of providing intuitiveness and convenience of use to the operator to compare medical images

In addition, according to the present invention, by specifying the type of the imaging device and the patient through mount selection to proceed with imaging, it has an effect of minimizing an image capturing step to prevent the operator's mistakes and provide convenience of use.

In addition, according to the present invention, when data is not located in the desired location of the operator for various reasons such as setting of an imaging device, file loading policy, the operator's mistakes and the like, it has an effect of providing convenience of use to the operator to facilitate moving and copying of data between patients or imaging devices on one screen.

In addition, according to the present invention, by setting the position, size and number of the mounts for each patient and imaging device, it has an effect of providing convenience of use to the operator because it is possible to set a customized layout according to the treatment site and treatment plan that vary for each patient.

### BRIEF DESCRIPTION OF THE DRAWINGS

FIG. 1 is a block diagram of an apparatus for managing medical image data and treatment data according to an exemplary embodiment of the present invention.

FIGS. 2 and 3 are diagrams illustrating a display unit according to an exemplary embodiment of the present invention.

FIG. 4 is a diagram for describing a method of imaging a patient using the apparatus for managing medical image data and treatment data according to an exemplary embodiment of the present invention.

FIG. 5 is a diagram for describing a method of loading medical image data and treatment data using the apparatus for managing medical image data and treatment data according to an exemplary embodiment of the present invention.

FIGS. 6 and 7 are diagrams for describing a method of copying and moving thumbnails using the apparatus for managing medical image data and treatment data according to an exemplary embodiment of the present invention.

FIGS. 8 to 10 are diagrams for describing the operation of a screen configuration unit according to an exemplary embodiment of the present invention.

FIG. 11 is a flowchart of a method for managing medical image data and treatment data according to an exemplary embodiment of the present invention.

FIG. 12 is a flowchart of a method of imaging a patient using the apparatus for managing medical image data and treatment data according to an exemplary embodiment of the present invention.

FIG. 13 is a flowchart of a method of loading medical image data and treatment data using the apparatus for managing medical image data and treatment data according to an exemplary embodiment of the present invention.

FIG. 14 is a flowchart of a method of copying and moving thumbnails using the apparatus for managing medical image data and treatment data according to an exemplary embodiment of the present invention.

### DETAILED DESCRIPTION OF EXEMPLARY EMBODIMENTS

Hereinafter, exemplary embodiments of the present invention will be described in more detail with reference to the accompanying drawings. In this case, it should be noted that the same components in the accompanying drawings are indicated by the same reference numerals as much as possible. In addition, detailed descriptions of well-known functions and configurations that may obscure the gist of the present invention will be omitted.

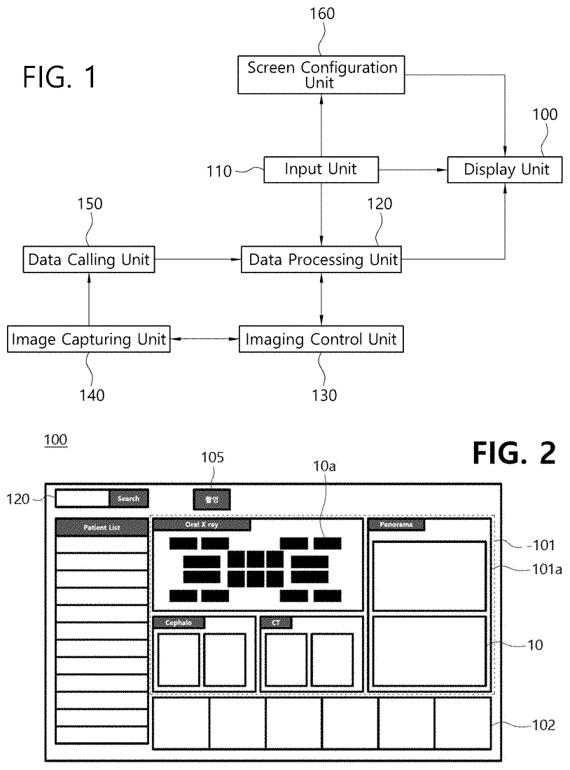

FIG. 1 is a block diagram of an apparatus for managing medical image data and treatment data according to an exemplary embodiment of the present invention.

As illustrated in FIG. 1, the apparatus for managing medical image data and treatment data according to an exemplary embodiment of the present invention may be configured by including a display unit 100, an input unit 110, a data processing unit 120 and a database 150.

The database 150 stores medical image data of a patient imaged by a plurality of imaging devices 140 and treatment data generated based on the medical image data.

Herein, a plurality of imaging devices 140 may include a plurality of 2D image capturing devices and 3D imaging devices. Specifically, the 2D image capturing device may be a variety of imaging devices for imaging a patient and generating a 2D medical image, including a Ceph (cephalometrics) imaging device. In addition, the 3D imaging device may be a variety of imaging devices for imaging a patient and generating a 3D medical image, including a computed tomography (CT) imaging device.

The medical image data may be 2D medical image data, such as Ceph, which is a pixel-format image, a panorama-format image, an image acquired by an IO sensor, an image acquired through X-ray and the like. In addition, it may be 3D medical image data such as an image in a volume format, an image in a Voxel format, an image acquired through CT imaging, an image acquired through MRI imaging and an image acquired through ultrasound imaging.

In addition, the treatment data may be treatment planning data and treatment device design data (e.g., design data of orthodontic devices, indirect attachment trays, jaw surgery wafers, dentures, etc.), and may be generated independently or in combination with medical image data.

The database 150 may generate a patient list by arranging medical image data and treatment data based on the patient's unique number, name and date of birth.

Meanwhile, although the database 150 has been described as one configuration of the apparatus for managing medical image data and treatment data according to an exemplary embodiment of the present invention, the present invention is not limited thereto, and the database 150 may be provided in an external server (not illustrated).

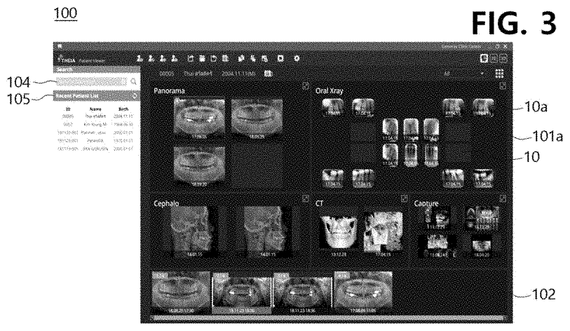

FIGS. 2 and 3 are diagrams illustrating a display unit according to an exemplary embodiment of the present invention.

The display unit 100 displays display data according to the operation of the apparatus for managing medical image data and treatment data according to an exemplary embodiment of the present invention. In particular, the display unit 100 displays a screen according to the execution and operation of an application, a web page and the like for establishing a treatment plan for a patient.

To this end, the display unit 100 may include a liquid crystal display (LCD), a light emitting diode (LED) display, an organic light emitting diode (OLED) display, a micro-electro mechanical system (MEMS) display and an electronic paper display. In addition, the display unit 100 may be combined with an input unit 110 to be implemented as a touch screen.

Herein, the input unit 110 is a configuration that generates input data in response to the operator's input, and may include a keypad, a dome switch, a touch panel, a jog & shuttle, a sensor, a touch key, a menu button and the like.

Referring to FIGS. 2 and 3, the display unit 100 is divided into a first display unit 101 and a second display unit 102, and the first display unit 101 may display thumbnails 10a of the patient's medical image data and treatment data and mounts 10 in which the thumbnails 10a are stored, and the second display unit 102 may display a patient search unit 104 and a patient list 105.

The data processing unit 120 generates thumbnails 10a of the patient's medical image data and treatment data stored in the database 150, and classifies and generates a plurality of mounts 10 for storing the thumbnails 10a for each imaging device 140. Also, the thumbnails 10a are stored in the plurality of mounts 10.

Accordingly, the display unit 100 displays a plurality of mounts 10 and the thumbnails 10a stored in the plurality of mounts 10 on a thumbnail view area 101.

Herein, if there are a plurality of medical image data and treatment data included in any one mount 10, the data processing unit 120 may display representative data among the same as a thumbnail 10a, and if there is a precedence relationship in the generation time of a plurality of image data and treatment data included in any one mount 10, the most recently generated data may be displayed as a thumbnail 10a.

In addition, the date when the medical image data or treatment data is generated may be displayed on the mount 10, and the number of medical image data and treatment data included in the mount 10 may be displayed.

In addition, medical image data and treatment data included in the mount 10 may be changed through an interactive function such as a mouse scroll, a touch slide and the like on the mount 10.

The data processing unit 120 may generate the thumbnail 10a, but it reduces the capacity of medical image data and treatment data having a relatively large capacity, and may maintain the capacity of medical image data and treatment data having a relatively small capacity.

When the mount 10 of a specific imaging device is selected by the operator, the input unit 110 transmits a mount selection signal for the selected mount 10 to a data call unit (not illustrated).

When a mount selection signal is received, the data call unit (not illustrated) calls medical image data or treatment data included in the mount 10 selected from the database 150.

The data processing unit 120 arranges the medical image data and the treatment data called by the data calling unit (not illustrated) by the order of generation and displays the same on a history view area 102 of the first display unit 101.

As such, the apparatus for managing medical image data and treatment data according to an exemplary embodiment of the present invention classifies medical image data and treatment data for each imaging device 140 and displays the same as thumbnails 10a on the display unit 100, and thus, it is possible to comprehensively compare and analyze medical image data and treatment data for each imaging device 140 on one screen, thereby providing the operator with intuitiveness and convenience of use for comparing medical images.

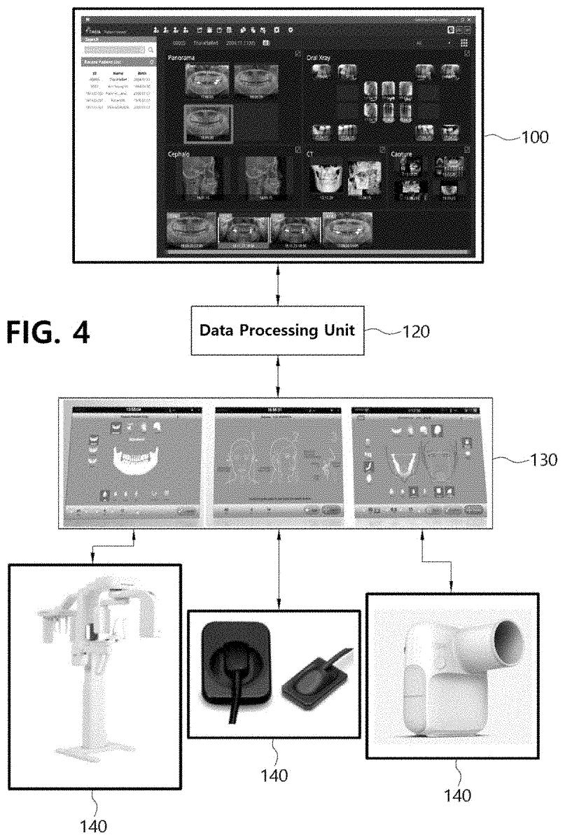

FIG. 4 is a diagram for describing a method of imaging a patient using the apparatus for managing medical image data and treatment data according to an exemplary embodiment of the present invention.

As illustrated in FIG. 4, the apparatus for managing medical image data and treatment data according to an exemplary embodiment of the present invention may further include an imaging control unit 130.

The operator selects a first selection mount from a plurality of mounts 10 through the input unit 110 and selects the imaging process of the imaging device 140. For example, the operator may first click a first selection mount and then click an imaging button 106 to proceed with imaging of the imaging device 140.

Accordingly, the input unit 110 transmits a first mount selection signal for the first selection mount and an imaging signal for imaging the patient to the data processing unit 120.

When the first mount selection signal and the imaging signal are received, the data processing unit 120 transmits imaging device information for the imaging device 140 including the first selection mount and patient information to be imaged to an imaging control unit 130.

The imaging control unit 130 receives imaging device information and patient information, and controls the imaging device 140 selected based on the imaging device information and the patient information to image the patient. Accordingly, the selected imaging device 140 generates medical image data by imaging the patient and transmits the same to the imaging control unit 130.

The imaging control unit 130 receives medical image data from the selected imaging device 140 and transmits the same to the data processing unit 120 and the database 150.

The data processing unit 120 generates a first thumbnail of the medical image data received from the imaging control unit 130, and stores the first thumbnail in the first selection mount.

In addition, the database 150 stores the medical image data received from the imaging control unit 130 as an original.

As such, the apparatus for managing medical image data and treatment data according to an exemplary embodiment of the present invention minimizes an image capturing step by specifying the type of the imaging device and the patient through mount selection, thereby preventing the operator's mistakes and providing convenience of use.

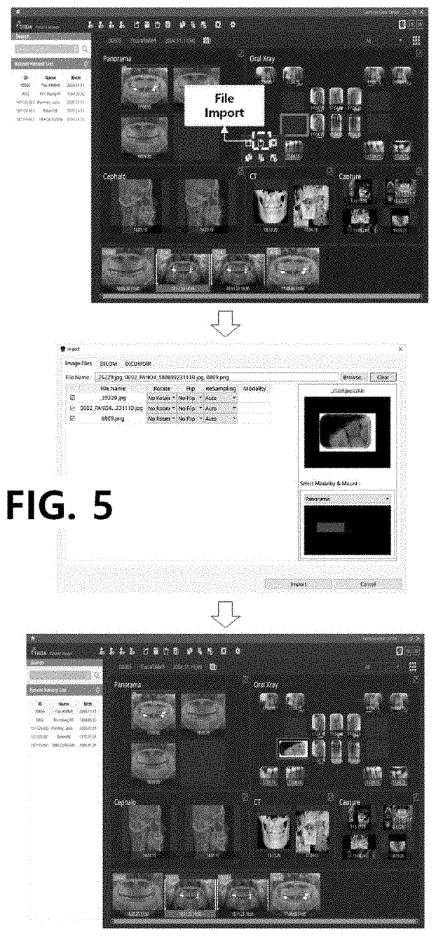

FIG. 5 is a diagram for describing a method of loading medical image data and treatment data using the apparatus for managing medical image data and treatment data according to an exemplary embodiment of the present invention.

The operator selects a second selection mount from a plurality of mounts 10 through the input unit 110 and selects load for the patient's medical image data and treatment data. For example, the operator first clicks the second selection mount and then clicks load to proceed with loading of the medical image data and the treatment data.

Accordingly, the input unit 110 transmits to the data processing unit 120 a second mount selection signal for the second selection mount and a loading signal for loading image data or treatment data.

The data processing unit 120 loads the medical image data or the treatment data from the database 150 when the second mount selection signal and the loading signal are received.

In addition, the data processing unit 120 generates a second thumbnail of the medical image data or treatment data loaded from the database 150, and stores the second thumbnail in the second selection mount.

Meanwhile, when the second mount selection signal and the loading signal are received, the data processing unit 120 may generate a list of medical image data and treatment data for each imaging device and display the same on the display unit 100. In this case, the operator may select medical image data and treatment data to be loaded from the list through the input unit 110.

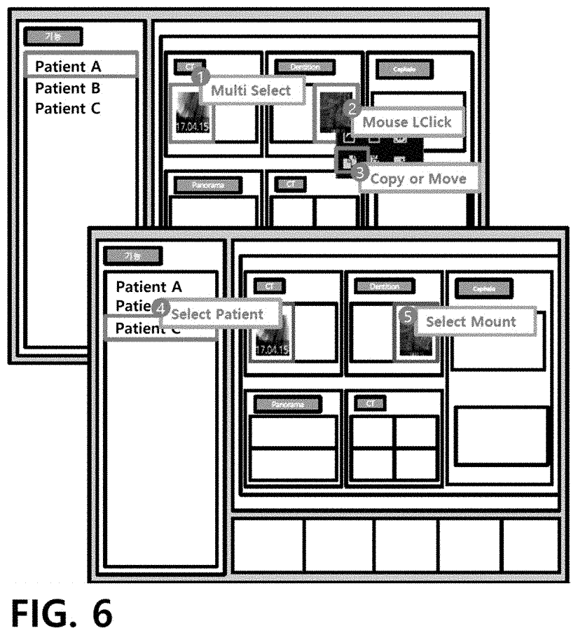

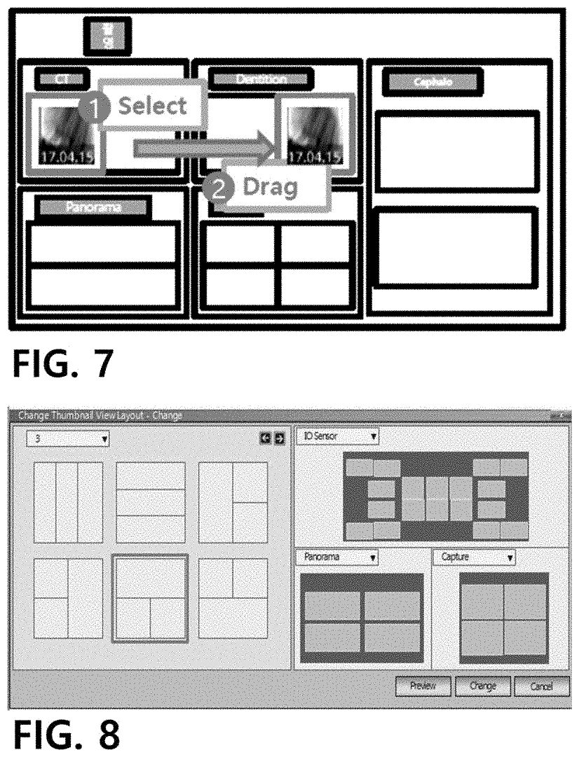

FIGS. 6 and 7 are diagrams for describing a method of copying and moving thumbnails using the apparatus for managing medical image data and treatment data according to an exemplary embodiment of the present invention.

Referring to FIG. 6, the operator selects a third selection mount that is a copy target from a plurality of mounts 10 through the input unit 110, selects copy or move for a third thumbnail stored in the third selection mount, selects a fourth selection mount that is a paste target from a plurality of mounts 10, and selects paste for the third thumbnail. For example, the operator first clicks a third selection mount, then clicks copy or move to save the third thumbnail, then clicks a fourth selection mount, and then clicks paste such that the third thumbnail may be pasted into the fourth selection mount.

Herein, the third and fourth selection mounts may be mounts 10 included in different imaging devices, and may be mounts 10 of different patients.

Accordingly, the input unit 110 transmits to the data processing unit 120 a third mount selection signal for a third selection mount, a fourth mount selection signal for a fourth selection mount and a copy signal for copying a third thumbnail stored in the third selection mount or a move signal for moving the third thumbnail and a paste signal for pasting the third thumbnail into a fourth mount.

When a third mount signal and a copy or move signal are received, the data processing unit 120 copies the third thumbnail stored in the third select mount, and when a fourth mount signal and a paste signal are received, it stores the third thumbnail in the fourth selection mount.

Referring to FIG. 7, the operator selects a third selection mount that is a copy target from a plurality of mounts 10 through the input unit 110, selects a fourth selection mount that is a moving target, and moves the third thumbnail into the fourth selection mount by dragging the third thumbnail stored in the third selection mount to a fourth selection mount.

Meanwhile, when the operator selects a mount 10, a toolbar for selecting any one of image, load and copy or move with respect to the selected mount 10 may be displayed on the display unit 100. In this case, the operator selects (clicks) a mount 10 and selects (clicks) any one of image, load and copy or move from the toolbar, and thus, the function of the apparatus for managing medical image data and treatment data according to the aforementioned exemplary embodiment of the present invention may be performed.

As such, when the data is not located in the desired location of the operator for various reasons, such as the setting of an imaging device, file loading policy, the operator's mistakes and the like, the apparatus for managing medical image data and treatment data according to an exemplary embodiment of the present invention may provide convenience of use to the operator because it may facilitate moving and copying of data between patients or imaging devices on one screen.

FIGS. 8 to 10 are diagrams for describing the operation of a screen configuration unit according to an exemplary embodiment of the present invention.

The apparatus for managing medical image data and treatment data according to an exemplary embodiment of the present invention may be configured by further including a screen configuration unit 160 for setting the position, size and number of the mounts 10 for each patient and imaging device.

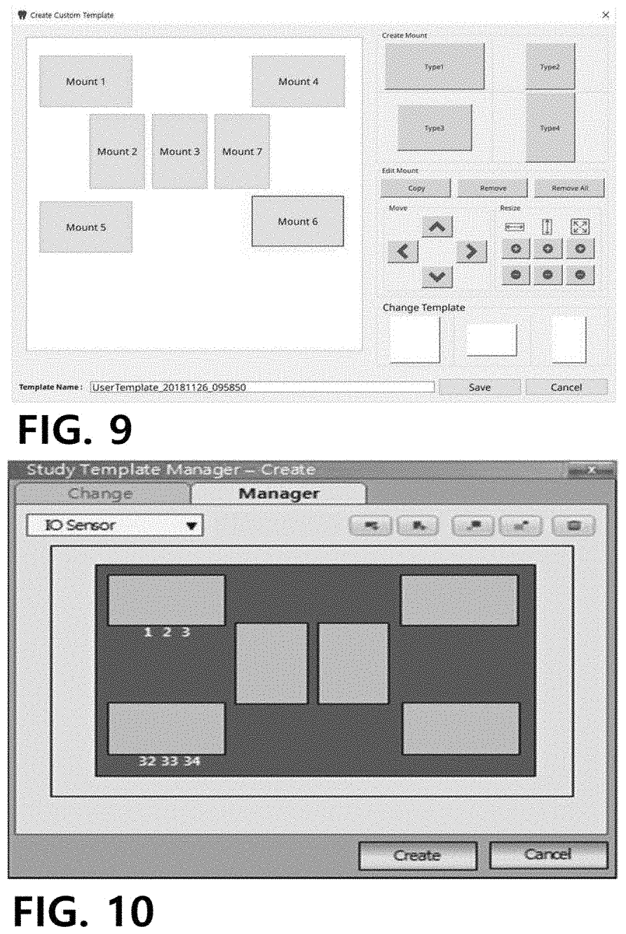

The screen configuration unit 160 may set the layout of a thumbnail view area 101. Specifically, the screen configuration unit 160 may set the number of imaging devices and the position, size and number of the mounts 10 for each imaging device (refer to FIG. 8), may set the position, size and number of the mounts 10 for each patient (refer to FIG. 9), and may set the position, size and number of the mounts 10 in a specific imaging device (refer to FIG. 10).

As such, by setting the position, size and number of the mounts 10 for each patient and imaging device through a screen configuration unit 160, the apparatus for managing medical image data and treatment data according to an exemplary embodiment of the present invention may provide convenience of use to the operator because it is possible to set a customized layout according to the treatment site and treatment plan that vary for patients.

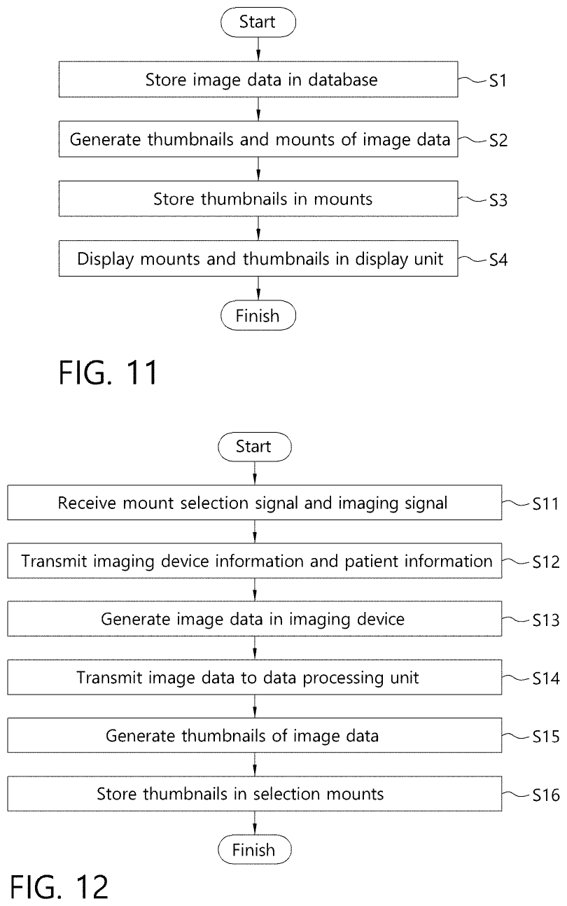

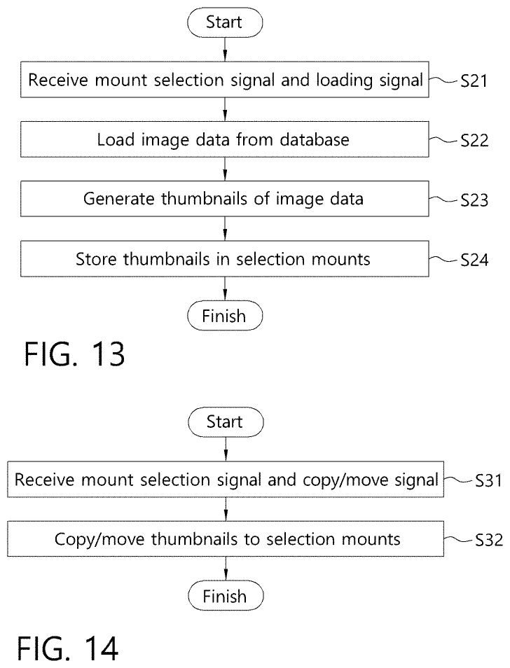

FIG. 11 is a flowchart of a method for managing medical image data and treatment data according to an exemplary embodiment of the present invention. FIG. 12 is a flowchart of a method of imaging a patient using the apparatus for managing medical image data and treatment data according to an exemplary embodiment of the present invention. FIG. 13 is a flowchart of a method of loading medical image data and treatment data using the apparatus for managing medical image data and treatment data according to an exemplary embodiment of the present invention. FIG. 14 is a flowchart of a method of copying and moving thumbnails using the apparatus for managing medical image data and treatment data according to an exemplary embodiment of the present invention.

Hereinafter, the method for managing medical image data and treatment data according to an exemplary embodiment of the present invention will be described with reference to FIGS. 1 to 14, but the same contents as in the apparatus for managing medical image data and treatment data according to an exemplary embodiment of the present invention will be omitted.

Referring to FIG. 11, the method for managing medical image data and treatment data according to an exemplary embodiment of the present invention may be configured by including the steps of storing medical image data in a database 150 S1, generating a thumbnail 10a and a mount 10 of medical image data S2, storing the thumbnail 10a in the mount 10 S3, and displaying the mount 10 and the thumbnail 10a on a display unit 100.

Specifically, first, medical image data of a patient imaged by a plurality of imaging devices 140 and treatment data generated based on the medical image data are stored in the database 110 S1.

Next, thumbnails 10a of the patient's medical image data and treatment data stored in the database 150 are generated, and a plurality of mounts 10 for storing the thumbnails 10a are classified and generated for each imaging device S2. Then, the thumbnails 10a are stored in the plurality of mounts 10 S3.

Next, the plurality of mounts 10 and the thumbnails 10a stored in the plurality of mounts 10 are displayed on a thumbnail view area 101 of a display unit 100 S4.

As such, the method for managing medical image data and treatment data according to an exemplary embodiment of the present invention classifies medical image data and treatment data for each imaging device 140 and displays the same as thumbnails 10a on the display unit 100, and thus, it is possible to comprehensively compare and analyze medical image data and treatment data for each imaging device 140 on one screen, thereby providing the operator with intuitiveness and convenience of use for comparing medical images.

Referring to FIG. 12 , the method for imaging a patient using the apparatus for managing medical image data and treatment data according to an exemplary embodiment of the present invention may be configured by including the steps of receiving a mount selection signal and an imaging signal S11, transmitting imaging device information and patient information S12, generating medical image data in an imaging device S13, transmitting medical image data to a data processing unit 120 S14, generating a thumbnail 10a of the medical image data, and storing the thumbnail 10a in the selection mount 10.

Specifically, first, a first mount selection signal for a first selection mount selected from a plurality of mounts and an imaging signal for imaging a patient are received S11.

Next, the imaging device information for the imaging device 140 including the first selection mount is transmitted S12. Then, the imaging device information is received, and the imaging device 140 selected based on the imaging device information is controlled to image the patient.

Next, the selected imaging device 140 generates medical image data by imaging the patient S13, and transmits the generated medical image data to the data processing unit 120 S14.

Next, a first thumbnail of the received medical image data is generated S15, and the first thumbnail is stored in the first select mount S16.

As such, the method for managing medical image data and treatment data according to an exemplary embodiment of the present invention minimizes an image capturing step by specifying the type of the imaging device and the patient through mount selection, thereby preventing the operator's mistakes and providing convenience of use.

Referring to FIG. 13, a method of loading medical image data and treatment data using the apparatus for managing medical image data and treatment data according to an exemplary embodiment of the present invention may be configured by including the steps of receiving a mount selection signal and a loading signal S21; loading medical image data from a database 150 S22, generating a thumbnail of the medical image data S23, and storing the thumbnail in a selection mount S24.

Specifically, first, a second mount selection signal for a second selection mount selected from a plurality of mounts 10 and a loading signal for loading image data or treatment data are received S21.

Next, medical image data or treatment data is loaded from the database 150 according to the loading signal S22.

Next, a second thumbnail of the medical image data or the treatment data loaded from the database 150 is generated S23, and the second thumbnail is stored in the second selection mount S24.

Referring to FIG. 14, a method of copying and moving a thumbnail using the apparatus for managing medical image data and treatment data according to an exemplary embodiment of the present invention may be configured by including the steps of receiving a mount selection signal and a copy signal or a move signal S31 and copying or moving the thumbnail 10a to a selection mount S32.

Specifically, first, when a third mount selection signal for a third selection mount selected from a plurality of mounts 10 and a copy signal for copying the third thumbnail stored in the third selection mount or a move signal for moving the third thumbnail are received S31, the third thumbnail is stored S32.

Next, when a fourth mount selection signal for a fourth selection mount selected from a plurality of mounts 10 and a paste signal for pasting the third thumbnail are received S32, the third thumbnail is stored in the fourth mount S32.

As such, when the data is not located in the desired location of the operator for various reasons, such as the setting of an imaging device, file loading policy, the operator's mistakes and the like, the method for managing medical image data and treatment data according to an exemplary embodiment of the present invention may provide convenience of use to the operator because it may facilitate moving and copying of data between patients or imaging devices on one screen.

The method for managing medical image data and treatment data according to an exemplary embodiment of the present invention may be configured by further including the step of setting the position, size and number of the mounts 10 for each patient and imaging device.

As such, by setting the position, size and number of the mounts 10 for each patient and imaging device through a screen configuration unit 160, the method for managing medical image data and treatment data according to an exemplary embodiment of the present invention may provide convenience of use to the operator because it is possible to set a customized layout according to the treatment site and treatment plan that vary for patients.

Meanwhile, the exemplary embodiments of the present invention disclosed in the present specification and drawings are only presented as specific examples to easily explain the technical contents of the present invention and help the understanding of the present invention, and are not intended to limit the scope of the present invention. That is, it will be apparent to those of ordinary skill in the art, to which the present invention pertains, that other modified examples may be implemented based on the technical spirit of the present invention.

The apparatus and method for managing medical image data and treatment data according to the present invention may be used in various fields, such as computing devices and the like, which are used to manage, process and analyze image data.

What is claimed is:

1. An apparatus for managing medical image data and treatment data, comprising:
a database for storing medical image data of a patient imaged by a plurality of imaging devices and treatment data generated based on the medical image data;
a data processing unit for generating thumbnails of the medical image data and the treatment data, and classifying and generating a plurality of mounts for storing the thumbnails for each imaging device; and
a display unit for displaying the plurality of mounts and the thumbnails stored in the plurality of mounts.

2. The apparatus of claim 1, wherein when a first mount selection signal for a first selection mount selected from the plurality of mounts and an imaging signal for imaging the patient are received, the data processing unit transmits imaging device information for the imaging device comprising the first selection mount.

3. The apparatus of claim 2, further comprising an imaging control unit for receiving the imaging device information and controlling the imaging device selected based on the imaging device information to image the patient.

4. The apparatus of claim 3, wherein the imaging control unit receives the medical image data generated by the selected imaging device imaging the patient and transmits the same to the data processing unit and the database.

5. The apparatus of claim 4, wherein the data processing unit generates a first thumbnail of the medical image data received from the imaging control unit, and stores the first thumbnail in the first selection mount.

6. The apparatus of claim 1, wherein when a second mount selection signal for a second selection mount selected from the plurality of mounts and a loading signal for loading the image data or treatment data are received, the data processing unit loads the medical image data or the treatment data from the database.

7. The apparatus of claim 6, wherein the data processing unit generates a second thumbnail of the medical image data or the treatment data loaded from the database, and stores the second thumbnail in the second selection mount.

8. The apparatus of claim 1, wherein when a third mount selection signal for a third selection mount selected from the plurality of mounts and a copy signal for copying a third thumbnail stored in the third selection mount or a move signal for moving the third thumbnail are received, the data processing unit stores the third thumbnail, and when a fourth mount selection signal for a fourth selection mount selected from the plurality of mounts and a paste signal for pasting the third thumbnail are received, the data processing unit stores the third thumbnail in the fourth mount.

9. The apparatus of claim 1, further comprising a screen configuration unit for setting the position, size and number of the mounts for each patient and imaging device.

10. A method for managing medical image data and treatment data, comprising:
storing medical image data of a patient imaged by a plurality of imaging devices and treatment data generated based on the medical image data in a database;
generating thumbnails of the medical image data and the treatment data, and classifying and generating a plurality of mounts for storing the thumbnails for each imaging device;
storing the thumbnails in the plurality of mounts; and
displaying the plurality of mounts and the thumbnails stored in the plurality of mounts.

11. The method of claim 10, further comprising:
receiving a first mount selection signal for a first selection mount selected from the plurality of mounts and an imaging signal for imaging the patient;
transmitting imaging device information for the imaging device comprising the first selection mount;
receiving the imaging device information and controlling the imaging device selected based on the imaging device information to image the patient;
transmitting the medical image data generated by imaging the patient by the selected imaging device; and
generating a first thumbnail of the received medical image data and storing the first thumbnail in the first selection mount.

12. The method of claim 10, further comprising:
receiving a second mount selection signal for a second selection mount selected from the plurality of mounts and a loading signal for loading the image data or treatment data;
loading the medical image data or the treatment data from the database according to the loading signal; and
generating a second thumbnail of the medical image data or the treatment data loaded from the database, and storing the second thumbnail in the second selection mount.

13. The method of claim 10, further comprising:
storing a third thumbnail when a third mount selection signal for a third selection mount selected from the plurality of mounts and a copy signal for copying the third thumbnail stored in the third selection mount or a move signal for moving the third thumbnail are received; and
storing the third thumbnail in a fourth mount when a fourth mount selection signal for a fourth selection mount selected from the plurality of mounts and a paste signal for pasting the third thumbnail are received.

14. The method of claim 10, further comprising setting the position, size and number of the mounts for each patient and imaging device.
